(19) United States
(12) Patent Application Publication
(10) Pub. No.: US 2024/0392097 A1
(43) Pub. Date: Nov. 28, 2024

(54) CASCADE DEGRADATION AND UPCYCLING OF POLYSTYRENE WASTE TO HIGH VALUE CHEMICALS

(71) Applicant: Virginia Tech Intellectual Properties, Inc., Blacksburg, VA (US)

(72) Inventors: Guoliang LIU, Blacksburg, VA (US); Zhen XU, Blacksburg, VA (US)

(21) Appl. No.: 18/695,210

(22) PCT Filed: Sep. 24, 2022
(86) PCT No.: PCT/US2022/044639

§ 371 Date: Mar. 25, 2024

Related U.S. Application Data
(60) Provisional application No. 63/248,158, filed on Sep. 24, 2021.

(51) Int. Cl. C08J 11/20; C08J 3/205

(52) U.S. Cl. CPC C08J 11/20; C08J 3/205; C08J 2325/06

(57) ABSTRACT

A method of converting polystyrene to diphenylmethane using either UV light and or heat and a Lewis acid is disclosed.

### CROSS REFERENCE TO RELATED APPLICATIONS

This application claims priority to and the benefit of co-pending U.S. provisional application No. 63/248,158, filed Sep. 24, 2021, entitled “ONE ORDER OF MAGNITUDE VALORIZATION OF POLYSTYRENE WASTE,” the contents of which are incorporated by reference herein.

### STATEMENT REGARDING FEDERALLY SPONSORED RESEARCH OR DEVELOPMENT

This invention was made with government support under contract DMR-1752611 awarded by the National Science Foundation. The government has certain rights in the invention.

### BACKGROUND

Based on the law of entropy, the vast bulk of polymer wastes deposited to the natural environment will eventually reach every corner of the planet, threatening the environment and inhabitants of the earth. Because the natural degradation of polymers is extremely slow, to mitigate the challenge, significant efforts have been dedicated to design and synthesize biodegradable polymers. The over 5000 million tons of commodity polymer wastes that humankind have accumulated to date, as well as the over 400 million tons being added annually,¹³ however, cannot wait for hundreds of years to natural degrade but urgently demand a solution to minimize the ramifications. Beyond mechanical and physical methods for recycling, polyethylene (PE),¹⁴ polyethylene terephthalate (PET) and polyesters have witnessed advances in their degradation and conversion into chemicals such as monomers and aromatics, but the recycling of other major commodity polymers remains gloomy. Polystyrene (PS) is widely used in packaging, appliances, automobile parts, toys, and gardening pots, and yet less than 10% of the PS produced each year (e.g., 15.6 million tons in 2020, GlobalData) is recycled. Like other polymers, a bottleneck in PS recycling is the financial incentive-recycling the polymer waste is more expensive than making virgin polymers due to the crude oil price and the low value of the recycled products (e.g., monomers, oligomers, fuels, waxes, and common aromatics). Existing processes to recycle PS cannot cover the capital investment if not subsidized by governments via policies and financial incentives, thus diming the attractiveness and potential of recycling. Upcycling and valorization, however, may provide unique opportunities even under free market conditions.

Compared with PE that can be converted to aromatics under high temperatures using precious metal catalysts for ring-formation and dehydrogenation, PS is intrinsically rich in phenyl groups (74 wt. %) and thus, in principle, is highly attractive for recovery and upcycling directly to aromatics. Laboratory-scale catalytic pyrolysis, usually conducted at temperatures above 350° C., can depolymerize PS into oligomers and sometimes styrene, benzene, toluene, and other aromatics.

### SUMMARY

In accordance with the purpose(s) of the present disclosure, as embodied and broadly described herein, the disclosure, in one aspect, includes a tandem degradation-upcycling process that photochemically and/or thermally degrades PS into aromatics over AlCl₃, then is followed by reaction with an electrophile, e.g., dichloromethane (DCM), to produce a high-value product, e.g., diphenylmethane (DPM). Compared to methods that require specific precursors (e.g., aromatics with certain functional groups), expensive catalysts, and often elevated temperatures, the present disclosure includes mild reaction conditions are mild, e.g., from ambient temperature to slightly elevated temperature and/or atmospheric pressure.

Other systems, methods, features, and advantages of the present disclosure will be or become apparent to one with skill in the art upon examination of the following drawings and detailed description. It is intended that all such additional systems, methods, features, and advantages be included within this description, be within the scope of the present disclosure, and be protected by the accompanying claims. In addition, all optional and preferred features and modifications of the described embodiments are usable in all aspects of the disclosure taught herein. Furthermore, the individual features of the dependent claims, as well as all optional and preferred features and modifications of the described embodiments are combinable and interchangeable with one another.

Additional advantages of the invention will be set forth in part in the description which follows, and in part will be obvious from the description, or can be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### BRIEF DESCRIPTION OF THE FIGURES

Many aspects of the present disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views.

FIG. 1 shows a table of results from different procedures of Example 2.

FIG. 2 generally shows panels a-g regarding the experimental setup and photographs of PS degradation; Panel 2a shows setups for degassing and degradation-upcycling; Panel 2b shows a mixture of AlCl₃, toluene, and PS in a three-neck quartz flask; Panel 2c shows the mixture in Panel 2b after stirring for ˜5 min; Panel 2d shows the mixture in Panel 2c after exposure to a UV lamp for ˜15 min; Panel 2e shows the mixture in Panel 2d after exposure to UV light for ˜5 h. Panel 2f shows a fraction of the light phase and heavy phase were transferred from Panel 2e to a colorimetric tube. Panel 2g shows the heavy phase in Panel 2f washed with acetone; Panel 2h shows a part of the heavy phase in Panel 2f washed with chloroform.

FIG. 3 shows SEC analysis of PS after varying lengths of degradation time and under varying reaction conditions. Graph 3a shows a graph of virgin PS and the product after photodegradation for 1, 2, 3, and 5 h. After 2, 3, and 5 h, the LS intensity became too weak to be detected and the traces were almost flat. Graph 3b shows PS degradation in control experiments. In T-w/o AlCl₃, the almost unchanged elution time indicate that PS was barely degraded. In T-w/o UV, the strong LS intensity but longer elution times show that PS was only partially degraded.

FIG. 4 shows a GC trace of the gas phase from the T-1 experiment, showing primarily benzene and toluene.

FIG. 5 shows a series of panels providing a characterization of acetone extract, grey precipitate, and solids residue. Panel 5a shows GC trace of the acetone solution in the T-1 experiment FIG. 2g. Panel 5b shows an SEC analysis of the acetone extract. Panels 5c and 5d show mass spectrometry analyses of the FIG. 5c acetone extract and FIG. 5d grey precipitate. FIG. 5e shows FTIR spectra of the grey precipitates, solid residue of FIG. 2 panel g and FIG. 2 panel h, respectively, and commercial AlCl₃.

FIG. 6 shows solvent participation during degradation.

FIG. 6a shows solvent mass change during PS degradation was determined in the experiments using toluene as the solvent and using DPM as an external reference for GC analysis. Three parallel experiments (T-react-1 to 3) were conducted. The changes in mass (2.2 wt. % on average) are attributed to both reacted and vaporized solvent. Therefore, the actual amount of solvent participated in the degradation was lower than our estimate.

FIG. 6b shows that as the volume of toluene was increased, ditolylmethanes became the more dominant product over benzyltoluenes, as evidenced by the decreasing α/β peak ratio.

FIG. 7 shows DPM upcycling as a function of time. FIG. 7a shows GC-FID traces after various lengths of reaction time for DPM upcycling. The traces were normalized based on the benzene solvent signal at 2.7 min. The intensity of the DPM signal (at 14.7 min) increased with reaction time. FIG. 7b shows the relationship between the upcycling reaction time and DPM yield. The dotted line represents the amount of benzene recycled from 1 g of PS under our reaction conditions. The mass of DPM was calculated using Equation 5. Based on the DCM feed ratio in photodegradation and upcycling in benzene, we estimate that ˜ 160 min was needed to upcycle all the benzene recycled from PS waste. FIG. 7c, FIG. 7d, and FIG. 7e show calibration curves of benzene, toluene, and diphenylmethane.

Additional advantages of the disclosure will be set forth in part in the description which follows, and in part will be obvious from the description, or can be learned by practice of the disclosure. The advantages of the disclosure will be realized and attained by means of the elements and combinations particularly pointed out in the appended claims. It is to be understood that both the foregoing general description and the following detailed description are exemplary and explanatory only and are not restrictive of the disclosure, as claimed.

### DETAILED DESCRIPTION

The disclosures herein will be described more fully hereinafter with reference to the accompanying drawings, in which some, but not all possible embodiments are shown. Indeed, disclosures may be embodied in many different forms and should not be construed as limited to the embodiments set forth herein; rather, these embodiments are provided so that this disclosure will satisfy applicable legal requirements. Like numbers refer to like elements throughout.

Many modifications and other embodiments disclosed herein will come to mind to one skilled in the art to which the disclosed compositions and methods pertain having the benefit of the teachings presented in the foregoing descriptions and the associated drawings. Therefore, it is to be understood that the disclosures are not to be limited to the specific embodiments disclosed and that modifications and other embodiments are intended to be included within the scope of the appended claims. Although specific terms are employed herein, they are used in a generic and descriptive sense only and not for purposes of limitation.

It is also to be understood that the terminology used herein is for the purpose of describing particular aspects only and is not intended to be limiting. As used in the specification and in the claims, the term “comprising” can include the aspect of “consisting of.” Unless defined otherwise, all technical and scientific terms used herein have the same meaning as commonly understood by one of ordinary skill in the art to which the disclosed compositions and methods belong. In this specification and in the claims which follow, reference will be made to several terms which shall be defined herein.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

Prior to describing the various embodiments, the following definitions are provided and should be used unless otherwise indicated.

### Definitions

Unless defined otherwise, all technical and scientific terms used herein have the same meaning as commonly understood by one of ordinary skill in the art to which this disclosure belongs. It will be further understood that terms, such as those defined in commonly used dictionaries, should be interpreted as having a meaning that is consistent with their meaning in the context of the specification and relevant art and should not be interpreted in an idealized or overly formal sense unless expressly defined herein.

As used herein, nomenclature for compounds, including organic compounds, can be given using common names, IUPAC, IUBMB, or CAS recommendations for nomenclature. When one or more stereochemical features are present, Cahn-Ingold-Prelog rules for stereochemistry can be employed to designate stereochemical priority, E/Z specification, and the like. One of skill in the art can readily ascertain the structure of a compound if given a name, either by systemic reduction of the compound structure using naming conventions, or by commercially available software, such as CHEMDRAW™ (Cambridgesoft Corporation, U.S.A.).

Alkyl may be generally lower alkyl, or C₁-C₆ alkyl. Examples of a C₁-C₆ alkyl group include, but are not limited to, methyl, ethyl, propyl, butyl, pentyl, hexyl, isopropyl, isobutyl, sec-butyl, tert-butyl, isopentyl, neopentyl, and isohexyl.

Halogen may be F, Cl, Br or I; or F, Cl, or Br; or F or Cl; or F; or Cl.

Haloalkyl may be generally lower haloalkyl, or C₁-C₆ haloalkyl, or C₁-C₃ alkyl. Examples of C₁-C₃ haloalkyl include CH₂F, CHF₂, CF₃, CH₂Cl, CHCl₂, CCl₃, CF₂CF₃, CF₂CF₂H, and CH₂CF₃.

The term “substituted” means that alkyl or aryl may be substituted by from one to five substituents which are fluorine, chlorine, bromine, iodine, C₁₋₁₂haloalkyl, nitro, C₁₋₁₂alkyl, C₅₋₁₂aryl or C₁₋₁₂alkoxyl, cyano, C₁₋₁₂haloalkoxyl, C₁₋₁₂alkylsulfenyl, C₁₋₁₂alkylsulfinyl, C₁₋₁₂alkylsulfonyl, C₁₋₁₂haloalkylsulfenyl, C₁₋₁₂haloalkylsulfinyl, or C₁₋₁₂haloalkylsulfonyl, hydroxyl, thiol, amino, oxo, carboxyl, carbonylalkylC₁₋₁₂acylamino, C₁₋₁₂alkoxy-carbonylamino, C₁₋₁₂haloalkoxycarbonylamino, C₁₋₁₂alkoxyimino, C₁₋₁₂haloalkoxyimino, or C₁₋₁₂alkylsulfonylamino, or sulfur pentafluoride; such substitution may be fluorine, chlorine, bromine, C₁₋₆haloalkyl C₁₋₆haloalkoxyl, oxo, carboxyl, carbonylalkyl and cyano.

The term leaving group means is understood by a person of ordinary skill as the a halide, e.g., fluoride, chloride, bromide, iodide, an alkylsulfonate e.g., methylsulfonate, trifluoromethylsulfonate an arylsulfonate, e.g, p-toluenesulfonate, hydroxide, alkoxide, aryloxide, carboxylate, e.g., acetate, ammonia, alkylamine and the like. Such leaving groups include the conjugate acids of the foregoing leaving groups.

As used herein, “comprising” is to be interpreted as specifying the presence of the stated features, integers, steps, or components as referred to, but does not preclude the presence or addition of one or more features, integers, steps, or components, or groups thereof. Moreover, each of the terms “by”, “comprising,” “comprises”, “comprised of,” “including,” “includes,” “included,” “involving,” “involves,” “involved,” and “such as” are used in their open, non-limiting sense and may be used interchangeably. Further, the term “comprising” is intended to include examples and aspects encompassed by the terms “consisting essentially of” and “consisting of.” Similarly, the term “consisting essentially of” is intended to include examples encompassed by the term “consisting of.

As used in the specification and the appended claims, the singular forms “a,” “an” and “the” include plural referents unless the context clearly dictates otherwise. Reference to “a” chemical compound refers one or more molecules of the chemical compound, rather than being limited to a single molecule of the chemical compound. Furthermore, the one or more molecules may or may not be identical, so long as they fall under the category of the chemical compound. Thus, for example, “a” polyamide is interpreted to include one or more polymer molecules of the polyamide, where the polymer molecules may or may not be identical (e.g., different molecular weights and/or isomers).

It should be noted that ratios, concentrations, amounts, and other numerical data can be expressed herein in a range format. Where the stated range includes one or both of the limits, ranges excluding either or both of those included limits are also included in the disclosure, e.g. the phrase “x to y” includes the range from ‘x’ to ‘y’ as well as the range greater than ‘x’ and less than ‘y’. The range can also be expressed as an upper limit, e.g. ‘about x, y, z, or less’ and should be interpreted to include the specific ranges of ‘about x’, ‘about y’, and ‘about z’ as well as the ranges of ‘less than x’, less than y′, and ‘less than z’. Likewise, the phrase ‘about x, y, z, or greater’ should be interpreted to include the specific ranges of ‘about x’, ‘about y’, and ‘about z’ as well as the ranges of ‘greater than x’, greater than y′, and ‘greater than z’. In addition, the phrase “about ‘x’ to ‘y”, where ‘x’ and ‘y’ are numerical values, includes “about ‘x’ to about ‘y”. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “about 0.1% to 5%” should be interpreted to include not only the explicitly recited values of about 0.1% to about 5%, but also include individual values (e.g., 1%, 2%, 3%, and 4%) and the sub-ranges (e.g., 0.5%, 1.1%, 2.4%, 3.2%, and 4.4%) within the indicated range.

As used herein, the terms “about,” “approximate,” “at or about,” and “substantially” mean that the amount or value in question can be the exact value or a value that provides equivalent results or effects as recited in the claims or taught herein. That is, it is understood that amounts, sizes, formulations, parameters, and other quantities and characteristics are not and need not be exact, but may be approximate and/or larger or smaller, as desired, reflecting tolerances, conversion factors, rounding off, measurement error and the like, and other factors known to those of skill in the art such that equivalent results or effects are obtained. In some circumstances, the value that provides equivalent results or effects cannot be reasonably determined. In such cases, it is generally understood, as used herein, that “about” and “at or about” mean the nominal value indicated ±10% variation unless otherwise indicated or inferred. In general, an amount, size, formulation, parameter or other quantity or characteristic is “about,” “approximate,” or “at or about” whether or not expressly stated to be such. It is understood that where “about,” “approximate,” or “at or about” is used before a quantitative value, the parameter also includes the specific quantitative value itself, unless specifically stated otherwise.

As used herein, the term “effective amount” refers to an amount that is sufficient to achieve the desired modification of a physical property of the composition or material. For example, an “effective amount” of a component refers to an amount that is sufficient to achieve the desired improvement in the property modulated by the formulation component, e.g. achieving the desired level of modulus. The specific level in terms of wt % in a composition required as an effective amount will depend upon a variety of factors

Unless otherwise specified, temperatures referred to herein are based on atmospheric pressure (i.e. one atmosphere).

As used herein, the terms “number average molecular weight” or “Mₙ” can be used interchangeably, and refer to the statistical average molecular weight of all the polymer chains in the sample and is defined by the formula:

$${M}_{n} = \frac{\sum {N}_{i} {M}_{i}}{\sum {N}_{i}} ,$$

where Mᵢ is the molecular weight of a chain and Ni is the number of chains of that molecular weight. Mₙ can be determined for polymers, e.g., polycarbonate polymers, by methods well known to a person having ordinary skill in the art using molecular weight standards, e.g. polycarbonate standards or polystyrene standards, preferably certified or traceable molecular weight standards.

Before proceeding to the Examples, it is to be understood that this disclosure is not limited to particular aspects described, and as such may, of course, vary. Other systems, methods, features, and advantages of foam compositions and components thereof will be or become apparent to one with skill in the art upon examination of the following drawings and detailed description. It is intended that all such additional systems, methods, features, and advantages be included within this description, be within the scope of the present disclosure, and be protected by the accompanying claims. It is also to be understood that the terminology used herein is for the purpose of describing particular aspects only and is not intended to be limiting. The skilled artisan will recognize many variants and adaptations of the aspects described herein. These variants and adaptations are intended to be included in the teachings of this disclosure and to be encompassed by the claims herein.

The disclosure includes the following aspects:

Aspect 1. A method of converting polystyrene to a compound of formula (I):

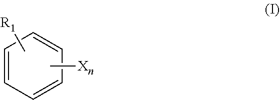

wherein R₁ is H, halogen, haloalkyl, or C₁-C₅ alkyl, SO₂, SO₃H;
X is selected from the group consisting of alkyl, substituted alkyl, haloalkyl, NO₂, NH₂, CN, C(O)alkyl wherein alkyl is substituted or unsubstituted, C(O)aryl, wherein aryl is substituted or unsubstituted, C(O)O-alkyl wherein alkyl is substituted or unsubstituted, CONHalkyl, C(O)N(alkyl)₂ wherein each alkyl is substituted or unsubstituted, C(O)NH(aryl), wherein aryl is substituted or unsubstituted, C(O)N(aryl)₂ wherein each aryl is substituted or unsubstituted, C(S)SH, C(S)S-alkyl wherein alkyl is substituted or unsubstituted, CH₂C(O)aryl wherein aryl is substituted or unsubstituted, CH₂C(O)N(alkyl)₂ wherein each alkyl is substituted or unsubstituted;
n is 0, 1, 2, or 3;
or a salt thereof;
comprising reacting a polystyrene of polymer unit (II):

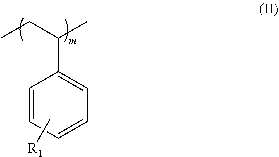

wherein m is an integer from 100 to 100000
with a Lewis acid in a solvent and irradiating the admixture with ultraviolet radiation to provide an irradiated polystyrene mixture comprising benzene and
treating the irradiated polystyrene mixture with an electrophile X-Y, wherein Y is a leaving group.

Aspect 2. The method of aspect 1 wherein the polystyrene is homopolymeric.

Aspect 3. The method of aspect 1 or 2 wherein the polystyrene is selected from atactic polystyrene, isotactic polystyrene, and syndiotactic polystyrene.

Aspect 4. The method of any one of the foregoing aspects wherein the polystyrene is a copolymerized polystyrene or a crosslinked polymer

Aspect 5. The method of any one of the foregoing aspects wherein the copolymerized polystyrene is a polystyrene-polycarbonate copolymer, a styrene-alkane copolymer, a styrene butadiene copolymer, or a styrene-maleic anhydride copolymer.

Aspect 6. The method of any one of the foregoing aspects wherein the copolymer is a graft copolymer.

Aspect 7. The method of any one of the foregoing aspects wherein the Lewis acid is selected from the group consisting of a Group I halide, a Group II dihalide, a transition metal halide, and a non-metal halide.

Aspect 8. The method of any one of the foregoing aspects wherein the Lewis acid is selected from the group consisting of lithium halides, sodium halides, potassium halides, manganese halides, calcium halides, scandium halides, titanium halides, manganese halides, iron halides, cobalt halides, nickel halides, copper halides, zinc halides, boron halides, aluminum halides, silver halides, tin halides, platinum halides, and palladium halides

Aspect 9. The method of any one of the foregoing aspects wherein the Lewis acid is an aluminum halide or a boron halide.

Aspect 10. The method of any one of the foregoing aspects wherein the Lewis acid is aluminum trichloride.

Aspect 11. The method of any one of the foregoing aspects wherein the Lewis acid is anhydrous or hydrated.

Aspect 12. The method of any one of the foregoing aspects wherein the solvent is an aromatic solvent.

Aspect 13, The method of any one of the foregoing aspects wherein the solvent is benzene, toluene, carbon disulfide, or an alkane such as hexanes.

Aspect 14. The method of any one of the foregoing aspects wherein X is alkyl.

Aspect 15. The method of any one of the foregoing aspects wherein X is COalkyl.

Aspect 16, The method of any one of the foregoing aspects wherein X is COaryl.

Aspect 17. The method of any one of the foregoing aspects wherein the solvent is anhydrous.

Aspect 18. The method of any one of the foregoing aspects wherein from about 60% to about 100%, or from about 70% to about 100%, or from about 80% to about 100%, or from about 60% to about 90%, or from about 70% to about 90%, or from about 80% to about 90% of the phenyl groups from the polystyrene are recovered from the polystyrene. It is understood that phenyl groups (or moieties) constitute about 74% by weight of a polystyrene. Thus, recovery of about 74 grams of a benzene group from about 100 grams of polystyrene may be considered the theoretical recovery from complete conversion.

Aspect 19. The method of any one of the foregoing aspects wherein the weight/weight (w/w) ratio of polystyrene to Lewis acid is from about 100:1 to about 1:100, or from about 80:1 to about 1:80 or from about 50:1 to about 1:50, or from about 20:1 to about 1:20, or from about 10:1 to about 1:10, or from about 5:1 to about 1:5, or from about 2:1 to about 1:2, or about 1:1.

Aspect 20. The method of any one of the foregoing aspects wherein the temperature may be from about room temperature to about 50° C. or from about 30° C. to about 50° C., or from about 35° C. to about 50° C., or from about 40° C. to about 50° C., or from about 30° C. to about 40° C., or from about less than 37° C. In another aspect, the reaction temperature may be about ambient temperature.

Aspect 21. A method of converting polystyrene to a compound of formula (I):

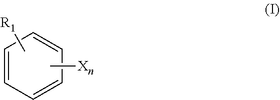

wherein R₁ is H, halogen, haloalkyl, or C₁-C₅ alkyl, SO₂, SO₃H;
X is selected from the group consisting of alkyl, substituted alkyl, haloalkyl, NO₂, NH₂, CN, C(O)alkyl wherein alkyl is substituted or unsubstituted, C(O)aryl, wherein aryl is substituted or unsubstituted, C(O)O-alkyl wherein alkyl is substituted or unsubstituted, CONHalkyl, C(O)N(alkyl)₂ wherein each alkyl is substituted or unsubstituted, C(O)NH(aryl), wherein aryl is substituted or unsubstituted, C(O)N(aryl)₂ wherein each aryl is substituted or unsubstituted, C(S)SH, C(S)S-alkyl wherein alkyl is substituted or unsubstituted, CH₂C(O)aryl wherein aryl is substituted or unsubstituted, CH₂C(O)N (alkyl)₂ wherein each alkyl is substituted or unsubstituted;
and n is 0, 1, 2, or 3;
or a salt thereof;
comprising reacting a polystyrene of polymer unit (II):

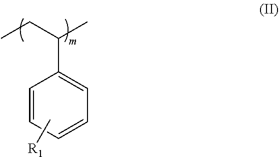

wherein m is an integer from 100 to 100000
with a Lewis acid in a solvent and heating the mixture to provide a polystyrene admixture comprising benzene and
treating the polystyrene admixture with an electrophile X-Y, wherein Y is a leaving group.

Aspect 22. The method of aspect 21 wherein the polystyrene is homopolymeric.

Aspect 23. The method of aspect 21 or 22 wherein the polystyrene is selected from atactic polystyrene, isotactic polystyrene, and syndiotactic polystyrene.

Aspect 24. The method of any one of aspects 21-23 wherein the polystyrene is a copolymerized polystyrene or a crosslinked polystyrene.

Aspect 25. The method of any one of aspects 21-24 wherein the copolymerized polystyrene is a polystyrene-polycarbonate copolymer, a styrene-alkane copolymer, a styrene butadiene copolymer, or a styrene-maleic anhydride copolymer.

Aspect 26. The method of any one of aspects 21-25 wherein the copolymer is a graft copolymer.

Aspect 27. The method of any one of aspects 21-26 wherein the Lewis acid is selected from the group consisting of a Group I halide, a Group II dihalide, a transition metal halide, and a non-metal halide.

Aspect 28. The method of any one of aspects 21-27 wherein the Lewis acid is selected from the group consisting of lithium halides, sodium halides, potassium halides, manganese halides, calcium halides, scandium halides, titanium halides, manganese halides, iron halides, cobalt halides, nickel halides, copper halides, zinc halides, boron halides, aluminum halides, silver halides, tin halides, platinum halides, and palladium halides

Aspect 29. The method of any one of aspects 21-28 wherein the Lewis acid is an aluminum halide or a boron halide.

Aspect 30. The method of any one of aspects 21-29 wherein the Lewis acid is aluminum trichloride.

Aspect 31. The method of any one of aspects 21-30 wherein the Lewis acid is anhydrous or hydrated.

Aspect 32. The method of any one of aspects 21-31 wherein the solvent is an aromatic solvent.

Aspect 33, The method of any one of aspects 21-32 wherein the solvent is benzene, toluene, carbon disulfide, or an alkane such as hexanes.

Aspect 34. The method of any one of aspects 21-33 wherein X is alkyl.

Aspect 35. The method of any one of aspects 21-34 wherein X is COalkyl.

Aspect 36, The method of any one of aspects 21-35 wherein X is COaryl.

Aspect 37. The method of any one of aspects 21-36 wherein the solvent is anhydrous.

Aspect 38. The method of any one of aspects 21-37 wherein from about 60% to about 100%, or from about 70% to about 100%, or from about 80% to about 100%, or from about 60% to about 90%, or from about 70% to about 90%, or from about 80% to about 90% of the phenyl groups from the polystyrene are recovered from the polystyrene. It is understood that phenyl groups (or moieties) constitute about 74% by weight of a polystyrene. Thus, recovery of about 74 grams of a benzene group from about 100 grams of polystyrene may be considered the theoretical recovery from complete conversion.

Aspect 39. The method of any one of aspects 21-38 wherein the weight/weight (w/w) ratio of polystyrene to Lewis acid is from about 100:1 to about 1:100, or from about 80:1 to about 1:80 or from about 50:1 to about 1:50, or from about 20:1 to about 1:20, or from about 10:1 to about 1:10, or from about 5:1 to about 1:5, or from about 2:1 to about 1:2, or about 1:1.

Aspect 40. The method of any one of aspects 21-39 wherein the temperature may be from about room temperature to about 100° C. or from about 30° C. to about 90° C., or from about 30° C. to about 80° C., or from about 30° C. to about 70° C., or from about 30° C. to about 60° C., or from about 30° C. to about 50° C., or from about 30° C. to about 40° C.

### EXAMPLES

Now having described the aspects of the present disclosure, in general, the following Examples describe some additional aspects of the present disclosure. While aspects of the present disclosure are described in connection with the following examples and the corresponding text and figures, there is no intent to limit aspects of the present disclosure to this description. On the contrary, the intent is to cover all alternatives, modifications, and equivalents included within the spirit and scope of the present disclosure.

General Experimental Method: Benzene (≥99%, HPLC) and toluene (≥99%, HPLC) were purchased from Sigma-Aldrich and purified by molecular sieves (4 Å) before use. Polystyrene (PS, Sigma-Aldrich, Mw˜192 kDa, PDI=2.15), dichloromethane (DCM, Spectranalyzed®, Fisher Chemical), aluminum chloride (AlCl₃, ReagentPlus>99%, Sigma-Aldrich), acetone (Analytical pure, Acros Organics), and argon (Ar, 99.998% purity, Praxair) were used as received. Diphenylmethane (DPM, >99%, Sigma-Aldrich) was used as a standard for product calibration and an external reference for the solvent consumption analysis. PS foam (M_{w}˜152 kDa, PDI=2.55) was obtained from packaging without pretreatment.

Degradation process and characterization techniques. Photodegradation was conducted under a UV reactor (Rayonet® RPR-100) equipped with 12 light bulbs (peak wavelength, 253.7 nm). The maximum light intensity was 12.5 W cm⁻² and the maximum output power was 250 W. All gas chromatography (GC) analyses were performed on a 6890 GC equipped with a DB-5 capillary column (30 m long×250 μm I.D. with a film thickness of 0.25 μm) from J&W Scientific (Wilmington, DE) and a flame ionization detector (FID). The following operating parameters were used for each GC analysis:

| Injection Port Temp. | 280° | C. |
|---|---|---|
| Purge Valve | 3 | mL min⁻¹ |
| Purge Time | 1 | min |
| Total Flow | 11 | mL min⁻¹ |
| Constant Flow | 0.8 | mL min⁻¹ |
| Injection Volume | 1 μL, split 1:10 | |
| Column Oven Initial Temp. | 40° | C. |
| Column Oven Initial Time | 3 | min |
| Column Oven Ramp Rate | 10° C. min⁻¹ to 280° C. | |
| Column Oven Final Temp. | 280° | C. |
| Column Oven Final Time | 1 | min |

All Gas chromatography-mass spectrometry (GC-MS) analyses were performed on a 6890 GC equipped with a 5973 Mass Selective Detector (MSD) from Agilent (Wilmington, DE). The MS Wiley library was used to identify the peaks. Separations were performed using the same GC column at the same operation conditions. The MSD transfer line temperature was 260° C.

Mass spectrometry (MS) analysis was performed using an Agilent 6220 Accurate-Mass time-of-flight mass spectrometer with an ESI probe in a positive mode and Agilent 1200 HPLC system (HPLC/MS) using isocratic 70/30 MeOH/H2O+0.1% fluoroacetic acid as the solvent. Samples were dissolved and then injected directly into the MS using a 1367B Agilent Autosampler.

Size exclusion chromatography (SEC, multi-detector EcoSEC HLC-8320GPC) was utilized to determine number average molecular weight (Mₙ), weight average molecular weight (M_{w}), and polydispersity index (PDI). The instrument was equipped with two TSKgel SuperHM-H columns, a refractive index (RI) detector, and a multi-angle light scattering detector (MALS). The oven temperature was kept at 50° C. and the eluent flow rate was 0.5 mL min⁻¹. The mobile phase was DMF containing 0.05 M LiBr.

Fourier transform infrared spectroscopy (FTIR) was performed at room temperature using a PerkinElmer ATR-FTIR (model Spectrum 100) in the range of 4000-1000 cm⁻¹ with 256 scans at a resolution of 4 cm⁻¹.

X-ray photoelectron spectroscopy (XPS) spectra were conducted on a PHI VersaProbe III scanning XPS microscope with monochromatic Al Kα X-ray source (1486.6 eV). XPS spectra were acquired with 200 μm/50 W/15 kV X-ray settings and dual-beam charge neutralization.

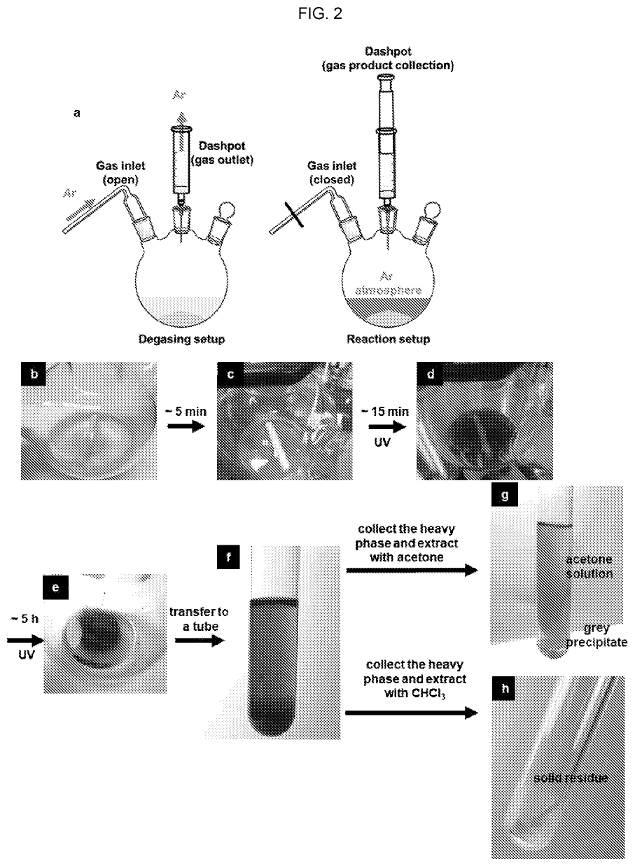

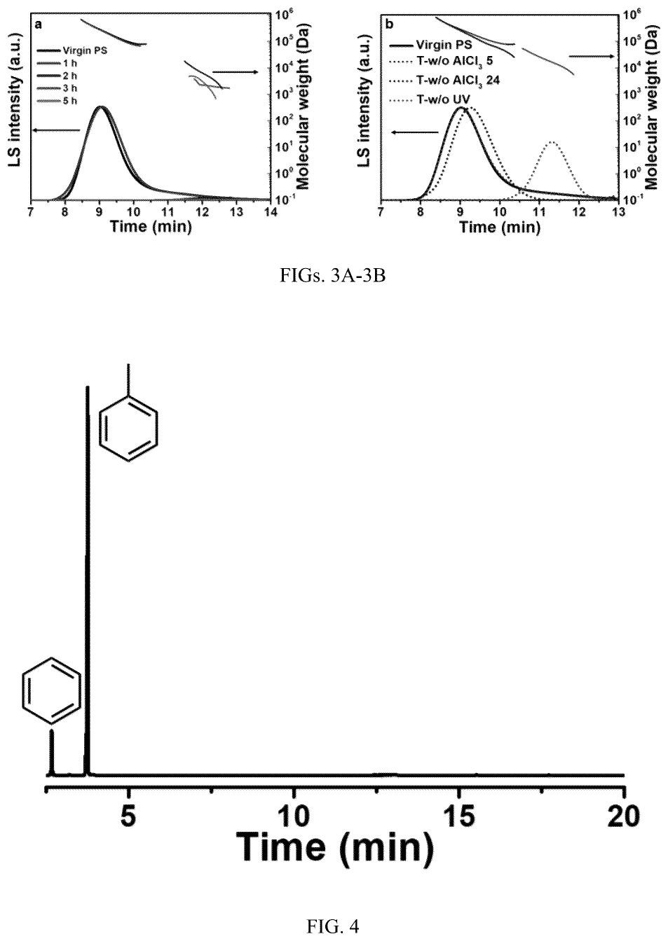

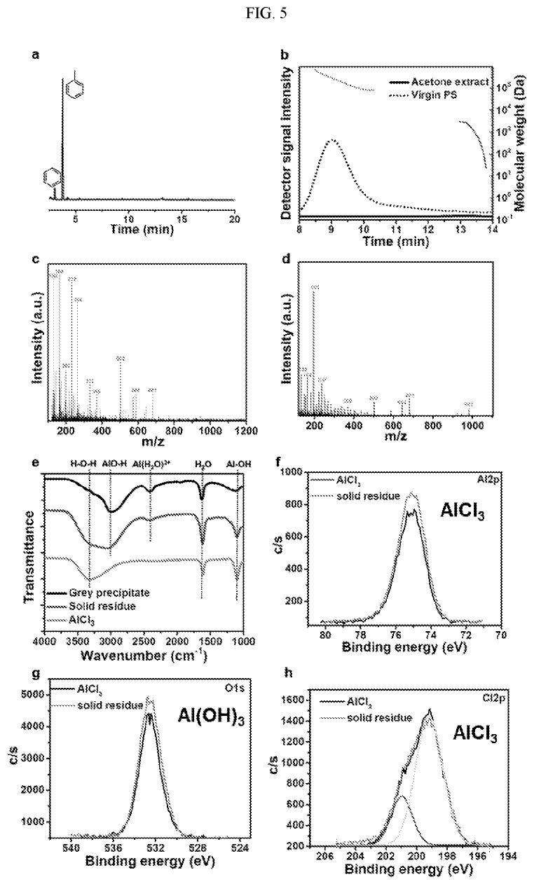

Molecular weight characterization as a function of time: PS (˜1.0 g, M_{w}˜ 192 kDa, Ð=2.15) was dissolved in toluene (˜ 10.0 mL) and combined with AlCl₃ (˜ 1.0 g) in a three-neck quartz flask (transmittance˜ 70% for 253.7 nm UV light) equipped with a stir bar, glass dashpot, gas inlet, and a glass stopper (FIG. 2, Panel 2a). The flask was placed under UV light to initiate the degradation. After 1, 2, 3, and 5 h of UV exposure, the light and heavy phases were sampled separately (50 μL each) using an automatic pipet and then combined for SEC characterization.

Control experiments for PS degradation under other conditions. T-w/o AlCl₃. PS (˜1.0 g, M_{w}˜192 kDa, Ð=2.15) was dissolved in toluene (˜10.0 mL) in a three-neck quartz flask (transmittance˜70% for 253.7 nm UV light) equipped with a stir bar, glass dashpot, gas inlet, and a glass stopper (FIG. 2, Panel 2a). After degassing with argon, the reactor was placed under UV light to initiate the degradation. After 5 and 24 h of UV exposure, the liquid phase was sampled for SEC characterization.

T-w/o UV. PS (˜1.0 g, M_{w}˜192 kDa, Ð=2.15) was dissolved in toluene (˜ 10.0 mL) and combined with AlCl₃ (˜ 1.0 g) in a three-neck quartz flask (transmittance˜70% for 253.7 nm UV light) equipped with a stir bar, glass dashpot, gas inlet, and a glass stopper (FIG. 2, Panel 2a). After degassing with argon, the reactor was placed in an oil bath at 43° C. to initiate the reaction. After 24 h, the light and heavy phases were sampled separately (50 μL each) using an automatic pipet and then combined for SEC characterization.

Characterization of the products from PS photodegradation. The analysis of the gas, light phase, and heavy phase products was performed as follows, using the GC/MS, SEC, FTIR, and TOF-MS procedures outlined previously. 1. Experimental setup and procedure. In a three-neck quartz flask (0.3 L, transmittance˜70% for 253.7 nm UV light) equipped with a stir bar, glass dashpot, gas inlet, and a glass stopper (FIG. 2a), PS (˜1.0 g, M_{w}˜192 kDa, Ð=2.15) was dissolved in a solvent (˜10.0 mL, benzene or toluene), followed by the addition of AlCl₃ (˜ 1.0 g). The total mass of the reactor and reactants were recorded (m₀). Immediately after loading, the reactor was purged with argon (20 mL s⁻¹ for 0.5 min and then 5 mL s⁻¹ for 15 min) to remove air in the flask. The reactor was sealed and the total mass was recorded again (m₁), from which the change in mass (m₁−m₀) attributed to the loss of solvent and replacement of atmosphere from air to argon was determined. To evaluate the loss of solvent during purging, the mass difference between air and argon were evaluated using their densities at 25° C. (in a 0.3 L flask containing ˜ 10 mL liquid, after replacing the air in the flask to argon, the mass increase was 132 mg). The loss of solvent was thus (132+m₀−m₁) mg. The quartz flask was then placed under a UV lamp (lamp working temperature˜37° C.) for 5 h.

2. Analysis of the gas phase. During degradation, the glass dashpot collected the gaseous products. The dashpot also prevented the reactor from over-pressurization. After 5 h, the gases in the dashpot were transferred to a glass cylinder and then immediately characterized using GC-MS. The total mass of the reactor was weighed again (m₂) to evaluate the mass of gas in the dashpot (m₂−m₁). The mass of the gas phase (gases in the flask and dashpot) was evaluated based on the mass in the dashpot and the volume ratio of gas in the dashpot and flask, excluding the mass of argon. The calculation results were showed in FIG. 2, Panel 2b.

The remaining products in the flask phase-separated into a light phase at the top and a heavy phase at the bottom. The total mass of the reactor was measured (m₃). To separate the light phase from the heavy phase, solvent (benzene or toluene, 10.0 mL) was added to the flask. The top phase was removed using a glass pipet. The procedure was repeated five times to ensure the light phase was fully removed. The potential residue was removed by a vacuum pump at room temperature, and the heavy phase remained because it is non-volatile. The mass of the reactor was weighted (m₄) to evaluate the recovery of the light phase (m₃−m₄) and heavy phase. The calculation results were shown in FIG. 2, Panel 2b.

Analysis of the heavy phase. A part of the heavy phase was transferred into a colorimetric tube for analysis in Step 5. The remainder of the heavy phase in the reactor was extracted by acetone (50.0 mL), forming a chartreuse acetone solution with insoluble precipitates. A part of this mixture was transferred into a colorimetric tube for illustration (FIG. 2, Panel 2g). The acetone solution was sampled (100 μL) and diluted by additional fresh acetone (˜2 mL) before characterization using GC/MS. Another ˜1 mL of the acetone solution was air-dried and dispensed into a DMF solution containing 0.05 M LiBr for SEC analysis. In addition, another ˜2 mL of the acetone solution was sampled for TOF-MS without treatment. After the sampling, the acetone solution was carefully decanted to obtain a grey precipitate. The grey precipitate was dried at 60° C. under reduced pressure to remove any volatiles. The insoluble precipitate was then analyzed by FTIR and TOF-MS.

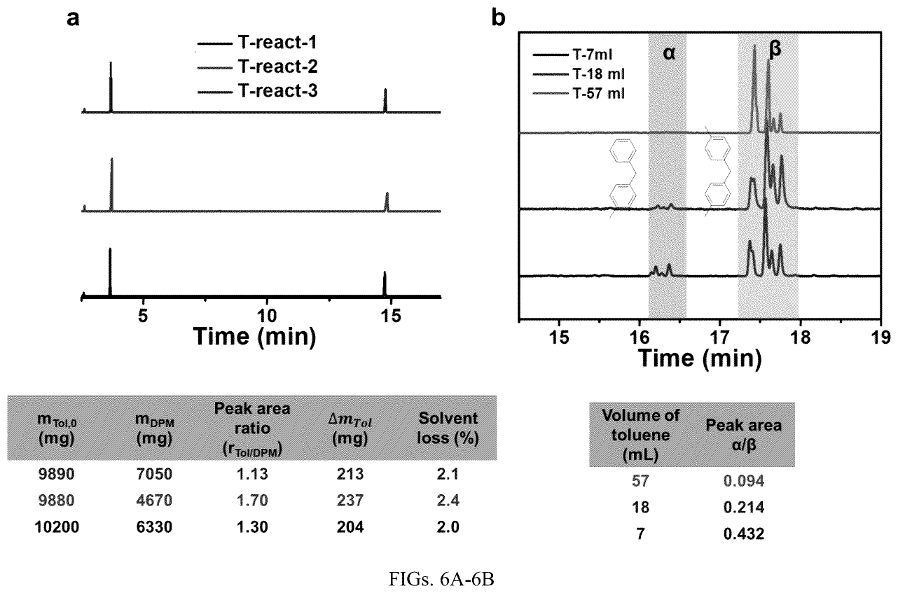

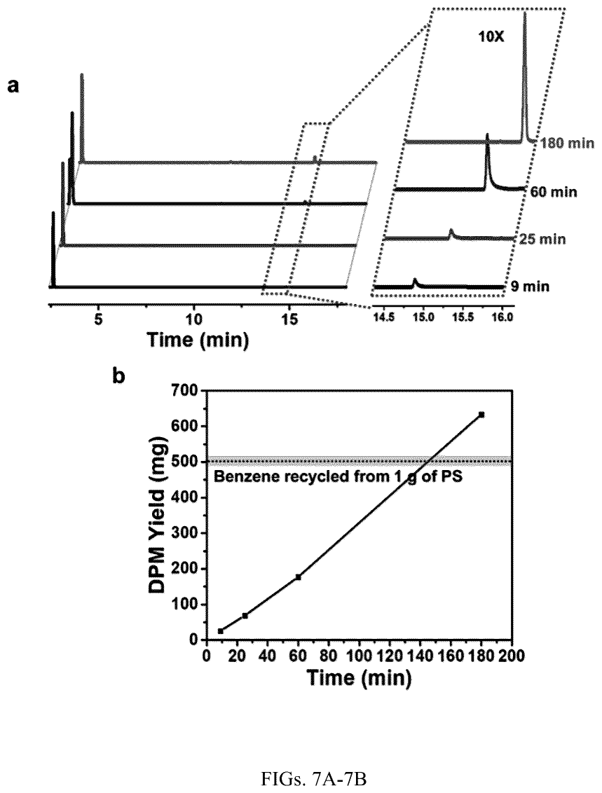

Solid residue in the heavy phase. A portion of the heavy phase was transferred to a colorimetric tube. Chloroform was injected into the colorimetric tube to dissolve the liquid components in the heavy phase. Because AlCl₃ has a lower solubility in chloroform than in acetone, AlCl₃ was preserved during solvent washing. A yellow solid residue (FIG. 2, Panel 2h.) was obtained after decanting the liquid. The remaining solid residue in the colorimetric tube was washed with chloroform (10 mL, 3 times) and then delivered for FTIR analysis (FIG. 5e).

With respect to FIG. 5, In the spectra of the grey precipitate, peaks above 3000 cm⁻¹ demonstrated the existence of hydroxides in the precipitate. The stretching vibration of octahedral aluminum hydrate (Al(H₂O)₆³⁺) occurred near 2411 cm⁻¹. Due to the hygroscopicity of hydroxides, water bending at 1626 cm⁻¹ were present in all samples.³¹ Peaks at 1103 cm⁻¹ were assigned to Al—OH bending.³² Therefore, the grey precipitate was primarily a mixture of aluminum hydroxides. The solid residue has similar spectrum as the commercial AlCl₃, therefore the solid residue may contain unreacted AlCl₃. (Panels 5f, 5g, 5h) The structure of the solid residue was further confirmed by XPS. The similar Al 2p and O 1s spectra indicated that solid residue and AlCl₃ have similar structures and are partially hydrolyzed. Cl 2p spectra further confirm the AlCl₃ in the solid residue, and the shoulder (201 eV) presented in both spectra may be caused by hydrolyzation.

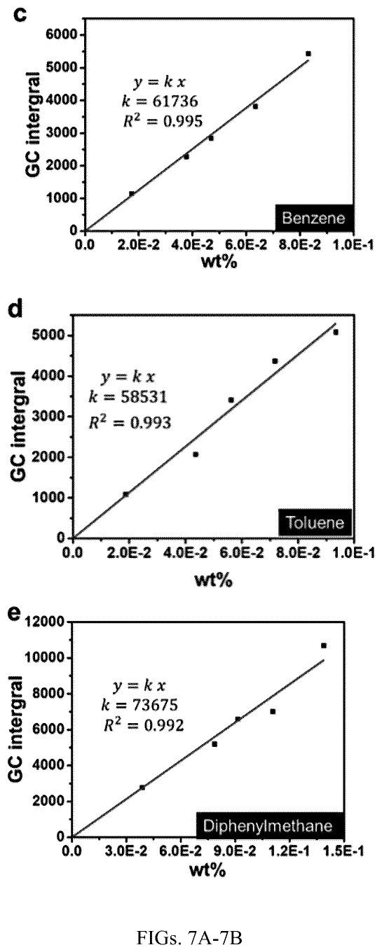

Determination of benzene yield from PS degradation. The experimental setup and preparation procedure were the same as the section above. After the degradation reaction, acetone (˜50 mL) was added to extract the organic products. In the experiments using toluene as the solvent (T-1 and T-2), a solution was sampled (˜0.5 mL) from the sealed reactor using a syringe equipped with a long needle. The sample solution was diluted with fresh acetone (˜5.0 mL), filtered to remove precipitates, and then delivered for GC and GC-MS analysis. The mass of benzene was calculated using equation (1) based on the ratio of areas of the benzene (˜2.7 min) and toluene (˜3.7 min) peaks in the GC traces.

$${m}_{B e n} = \frac{r}{{k}_{B e n} / {k}_{T o l}} \times {m}_{Tol , 0} \qquad ( 1 )$$

where r is the peak area ratio of benzene over toluene; m_{Ben} is the mass of benzene in solution; m_{Tol,0} is the mass of toluene solvent in the reactor; k is the slope of the GC calibration curve (FIGS. 7c-e, k_{Tol}=58531 and k_{Ben}=61736).

The amount of benzene in gas phase was evaluated using equation (2) based on Raoult's law and the ideal gas equation,

$${m}_{B e n , g} = \frac{c \times {P}_{B e n}^{*} \times V}{R T} \times {m}_{B e n} \qquad ( 2 )$$

where P*_{Ben} is the saturated vapor pressure of benzene (13.33 kPa) at the post-reaction temperature (i.e., 20° C.); c is the molar concentration of benzene in the liquid phase; V is the gas volume in the reactor (˜0.29 L); R is the ideal gas constant; T is temperature (293 K); m_{Ben} is the molar mass of benzene; m_{Ben,g} is the mass of benzene vapor.

In the experiments using benzene as the solvent, the mass of benzene was determined differently due to the absence of toluene. The amount of acetone for extraction was accurately measured (m_{Ace}). After the sediment of the precipitate, an acetone sample solution was collected and diluted with fresh acetone (m_{Ace,d}) before GC analysis. Based on the GC calibration curve (FIG. 7c), the mass of benzene in the solution (m_{Ben,l}) was evaluated using Equation (3).

$${m}_{Ben , l} = \frac{( {A}_{B e n} / {k}_{B e n} ) \times ( {m}_{Ace , d} + {m}_{Ace , s} )}{{m}_{Ace , s} / ( {m}_{A c e} + {m}_{PS} + {m}_{Ben , 0} )} \qquad ( 3 )$$

where A_{Ben} is the integrated GC peak area of benzene, k is the slope of the GC calibration curve (FIG. 7c, k_{Ben}=61736), mace is the mass of acetone for extraction; m_{Ace,d} is the mass of acetone for dilution; m_{PS} is the mass of PS before degradation; m_{Ben,0} is the mass of solvent benzene before degradation and m_{Ace,s} is the mass of the acetone sample solution.

The mass of benzene in the gas phase was evaluated using equation (2). The molar concentration c of benzene was approximated to one for simplicity.

Determination of solvent participation during PS degradation.

To accurately determine the solvent loss during PS degradation, three parallel experiments were conducted. In each experiment, the procedure was as follows. PS (˜1.0 g, M_{w}˜192 kDa, Ð=2.15) was dissolved in toluene (˜10.0 mL), followed by the addition of AlCl₃ (˜1.0 g), in a three-neck quartz flask (transmittance˜70% for 253.7 nm UV light) equipped with a stir bar, glass dashpot, gas inlet, and a glass stopper (FIG. 2, Panel 2a). After 5 h of UV exposure, acetone (˜ 50 mL) was added to stop the degradation. Diphenylmethane (˜ 3.5 g) was measured in a clean glass vial. Subsequently, the vial containing diphenylmethane was added to the acetone solution and DPM was used as an external reference. After swirling and ultrasonication for ˜ 1 min, the acetone solution was sampled for GC. The amount of reacted toluene was calculated using equation (4).

$$Δ {m}_{T o l} = {m}_{Tol , 0} - \frac{r}{{k}_{T o l} / {k}_{D P M}} \times {m}_{D P M} \qquad ( 4 )$$

where Δm_{Tol} is the toluene reacted; r is the peak area ratio of toluene over diphenylmethane; m_{Tol,0} is the weighted mass of toluene solvent; m_{DPM} is the mass of diphenylmethane external reference; k is the slope of the GC calibration curves (see FIGS. 7d-e, k_{Tol}=58531 and k_{DPM}=73675).

Photodegradation of PS foam and upcycling in benzene. PS foam (˜1.0 g) was photodegraded in benzene (˜10.0 mL) using AlCl₃ (˜1.0 g) under UV light for 5 h. After photodegradation, DCM (5.0 mL) was injected to the reactor to convert the photodegradation products into DPM. Similarly, the liquid in the reactor phase-separated into two phases. The light liquid phase was sampled using a syringe (˜ 0.5 mL) equipped with a long needle at 9, 25, 60, and 180 min after the injection of DCM. Each sample was diluted with acetone (˜ 5.0 mL) and delivered for GC analysis. The yield of DPM was evaluated using Equation (5) based on the GC peak ratio of benzene (˜2.7 min) and DPM (˜ 14.7 min).

$${m}_{D P M} = \frac{r}{{k}_{D P M} / {k}_{B e n}} \times {m}_{B e n} \qquad ( 5 )$$

where r is the peak area ratio of diphenylmethane over benzene; m_{Ben} is the actual mass of benzene solvent; m_{DPM} is the mass of DPM; k is the slope of the GC calibration curve (k_{DPM}=73675 and k_{Ben}=61736) (See FIGS. 7c and 7e). The DPM vapor was ignored due to the low vapor pressure (<0.133 kPa) and high boiling point (264° C.).

Example 2 production of diphenylmethane through photochemical Deg-Up reaction In a three-neck quartz flask equipped with a stir bar, gas inlet, and glass stoppers, PS (˜1.0 g) is added and dissolved in benzene (˜3.0 mL), followed by the addition of AlCl₃ (˜ 0.2 g). Immediately after loading, the reactor was purged with argon (20 mL s⁻¹ for 0.5 min and then 5 mL s⁻¹ for 15 min) to remove air and moisture in the flask. The quartz flask was then placed under a UV lamp (working temperature˜37° C.) for the PS degradation. The degradation reaction is completed after 5 h, producing intermediate benzene from PS with high yield (70 wt. %). The intermediate benzene is then upcycled into diphenylmethane with dichloromethane (5 mL) for 3 h at room temperature. The reaction mixture is then quenched by ice. The upcycling products, including naphtha oils (˜10 wt. %), diphenylmethane (˜75 wt. %), and pitch oil (˜15 wt. %), are separated by distillation.

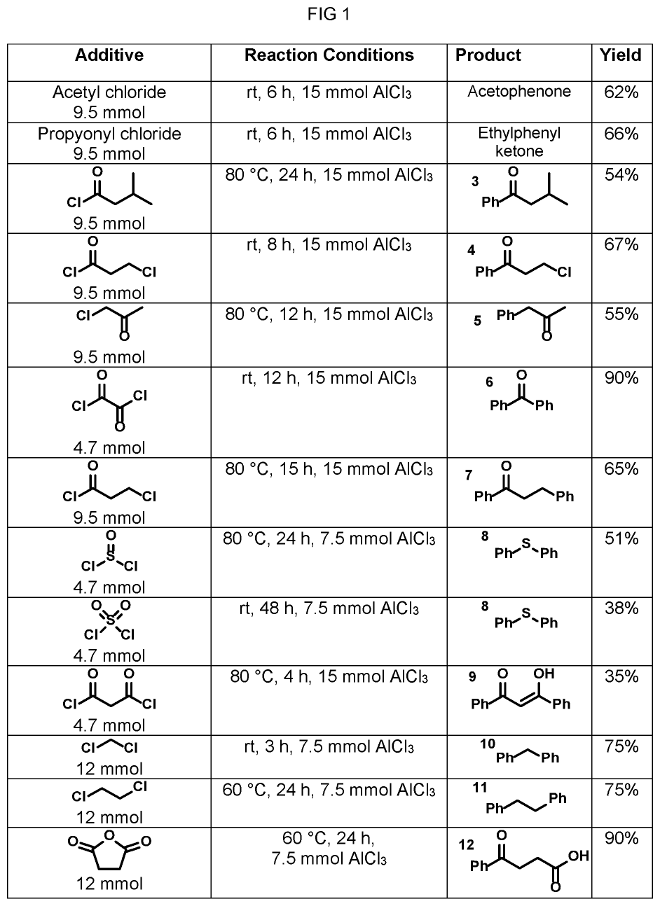

Example 3 production of diphenylmethane through thermal Deg-Up reaction In the thermal Deg-Up reaction, the reactor and reactors are prepared the same as Example 2. Immediately after reactants loading, the reactor was purged with argon (20 mL s⁻¹ for 0.5 min and then 5 mL s⁻¹ for 15 min) to remove air and moisture in the flask. The quartz flask was then placed into an oil bath at 100° C. for the PS degradation. The degradation reaction is completed after 5 h, producing intermediate benzene from PS with high yield (74 wt. %). The intermediate benzene is then upcycled into diphenylmethane with dichloromethane (5 mL) for 3 h at room temperature. The reaction mixture is then quenched by ice. The upcycling products, including naphtha oils (˜10 wt. %), diphenylmethane (˜75 wt. %), and pitch oil (˜15 wt. %), are separated by distillation. The products produced by the method of Example 2 are shown in FIG. 1.

It should be emphasized that the above-described embodiments of the present disclosure are merely possible examples of implementations set forth for a clear understanding of the principles of the disclosure. Many variations and modifications may be made to the above-described embodiment(s) without departing substantially from the spirit and principles of the disclosure. All such modifications and variations are intended to be included herein within the scope of this disclosure and protected by the following claims.

1. A method to convert polystyrene to diphenylmethane comprising admixing a polystyrene with an anhydrous Lewis acid in a substantially anhydrous solvent and irradiating the admixture with ultraviolet radiation to provide an irradiated polystyrene mixture and treating the irradiated polystyrene mixture with dichloromethane.

2. The method of claim 1 wherein the Lewis acid is aluminum trichloride.

3. The method of claim 1 wherein the solvent is benzene or toluene, or a mixture thereof.

4. The method of claim 1 wherein from about 50% to about 80%; or from about 50% to about 70%; or from about 60% to about 80% or from about 70% to about 80%; or from about 75% to about 80% of phenyl groups are recovered from the polystyrene as measured by benzene yield.

5. The method of claim 1 the aluminum trichloride and polystyrene are admixed in about a 1:1 weight ratio; or about 0.9:1; or about 1:0.9.

6. The method of claim 1 wherein the temperature is less than 37° C.

7. The method of claim 1 wherein the temperature is from about 33° C. to about 44° C.; or from about 33° C. to about 41° C.; or from about 35° C. to about 44° C.; or from about 35° C. to about 41° C.; or from about 37° C. to about 43° C.; or from about 37° C. to about 41° C.;

8. The method of claim 1 wherein the pressure is about atmospheric pressure.

9. The method of claim 2 wherein the solvent is benzene or toluene, or a mixture thereof.

10. The method of claim 2 wherein from about 50% to about 80%; or from about 50% to about 70%; or from about 60% to about 80% or from about 70% to about 80%; or from about 75% to about 80% of phenyl groups are recovered from the polystyrene as measured by benzene yield.

11. The method of claim 2 the aluminum trichloride and polystyrene are admixed in about a 1:1 weight ratio; or about 0.9:1; or about 1:0.9.

12. The method of claim 1 wherein the temperature is less than 37° C.

13. The method of claim 2 wherein the temperature is from about 33° C. to about 44° C.; or from about 33° C. to about 41° C.; or from about 35° C. to about 44° C.; or from about 35° C. to about 41° C.; or from about 37° C. to about 43° C.; or from about 37° C. to about 41° C.;

14. The method of claim 2 wherein the pressure is about atmospheric pressure.
